(19) United States
(12) Patent Application Publication
(10) Pub. No.: US 2018/0003644 A1
(43) Pub. Date: Jan. 4, 2018

(54) ASSAY, METHOD, DEVICE AND KIT FOR DETECTING ODOROUS COMPOUNDS

(71) Applicant: NOVOZYMES A/S, Bagsvaerd (DK)

(72) Inventor: Liv Spaanger Christiansen, Gentofte (DK)

(73) Assignee: NOVOZYMES A/S, Bagsvaerd (DK)

(21) Appl. No.: 15/542,820

(22) PCT Filed: Jan. 22, 2016
(86) PCT No.: PCT/EP2016/051329

§ 371 Date: Jul. 11, 2017

(30) Foreign Application Priority Data
Jan. 26, 2015 (EP) 15152538.3

(51) Int. Cl. G01N 21/78; C12Q 1/02

(52) U.S. Cl. CPC G01N 21/783; C12Q 1/02; G01N 2333/195

(57) ABSTRACT

The present invention concerns an assay, a method and a device for detecting the presence of an odorous compound on a surface comprising enclosing the surface in a compartment, which compartment comprises an odor capturing agent including a visual indicating agent, wherein the surface comprises an odorous compound derived from biofilm.

### FIELD OF THE INVENTION

The present invention concerns an assay for detecting the presence of odorous compound on a surface, a method for detecting the presence of odorous compound on a surface and a method for comparing the presence of odorous compound on a first surface with the presence of an odorous compound on at least one more surface. The invention further concerns a device and a kit for detecting for the presence of odorous compound.

### DESCRIPTION OF THE RELATED ART

When laundry items like T-shirts or sportswear are used, they are exposed to bacteria from the body of the user and from the rest of the environment in which they are used. Some of these bacteria are capable of adhering to the laundry item and form a biofilm on the item. The presence of bacteria implies that the laundry items become sticky and therefore odor adheres to the sticky areas.

When dirty laundry items are washed together with less dirty laundry items the dirt and odor present in the wash liquor tend to stick to the biofilm. As a result hereof the laundry item is more “soiled” after wash than before wash. Further, such bacteria in biofilm are a source of bad odor, which develops during and after use of the laundry item. The bad odor is difficult to remove and may remain even after wash. The reason for this bad odor is adhesion of bacteria and odorous molecules to the textile surface. Because of the adhesion to the textile, the bacteria may remain even after wash, and continue to be a source of bad odor.

Also, the interior of dishwashing machines or laundry washing machines may be subject to growth of biofilm. The growth and proliferation of microbes in a these machines generally occurs from exposure to prolonged warm, moist environments which may contain soap residue and clothing or food residues. This environment leads to the development of undesirable odors and biofilm. Biofilm growth further leads to degradation of the rubber which potentially results in reduced life cycle of the rubber parts or the entire washing machine as well as deposition of machine biofilm on the laundry items and dishware.

### SUMMARY OF THE INVENTION

The present invention concerns an assay for detecting the presence of odorous compound on a surface comprising enclosing the surface in a compartment, which compartment comprises an odor capturing agent including a visual indicating agent and wherein the surface comprises an odorous compound derived from biofilm.

The present invention further concerns a method for detecting the presence of odorous compound on a surface comprising enclosing the surface in a compartment, which compartment comprises an odor capturing agent including a visual indicating agent and wherein the surface comprises an odorous compound derived from biofilm.

Further is claimed a method for comparing the presence of odorous compound on a first surface with the presence of an odorous compound on at least one more surface comprising:

a. Enclosing the first surface in a first compartment comprising an odor capturing agent including a visual indicating agent;

b. enclosing at least one more surface in one or more separate compartments, where each compartment comprise an odor capturing agent including a visual indicating agent;

c. comparing the color development in each compartment.

The invention also concerns a device and a kit for detecting the presence of odorous compound on a surface.

### Definitions

Composition for cleaning hard surfaces: The term “composition for cleaning hard surfaces” refers to compositions intended for cleaning hard surfaces such as floors, tables, walls, roofs etc. as well as surfaces of hard objects such as cars (car wash) and dishes (dishware).

Dishware: The term dish ware is intended to mean any form of kitchen utensil used in domestic or industrial kitchen or food industry such as dinner set or tableware such as but not limited to pans, plates, cops, knives, forks, spoons, porcelain etc. The dishware can be made of any suitable material such as metal, glass, rubber, plastic, PVC, acrylics, ceramics, china or porcelain.

Dish washing composition: The term “dish washing composition” refers to compositions comprising detergent components, which composition is intended for cleaning dishes, table ware, glass ware, cutting boards, pots, pans, cutlery and all forms of compositions for cleaning hard surfaces areas in kitchens. The present invention is not restricted to any particular type of dish wash composition or any particular detergent component. The dish washing composition can be used for both domestic dish washing, industrial and institutional dish washing including composition for ADW.

Detergent Composition: The term “detergent composition” refers to compositions that find use in the removal of undesired compounds from surfaces to be cleaned, such as textile surfaces.

The detergent composition may be used to e.g. clean textiles for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; fabric fresheners; fabric softeners; and textile and laundry pre-spotters/pretreatment). The detergent formulation may contain one or more enzymes such as hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, beta-glucanases, arabinosidases, hyaluronidase, chondroitinase, laccase, DNase, chlorophyllases, amylases, perhydrolases, peroxidases, xanthanase and mixtures thereof. The detergent composition may further comprise detergent ingredients such as surfactants, builders, chelators or chelating agents, bleach system or bleach components, polymers, fabric conditioners, foam boosters, suds suppressors, dyes, perfume, tannish inhibitors, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

Hard surface: The term “Hard surface” is defined herein as hard surfaces including floors, tables, walls, roofs etc. as well as surfaces of hard objects such as cars (car wash) and dishes (dishware). The hard surface can be in a domestic house, industries or institutions.

Laundering: The term “laundering” relates to both household laundering and industrial laundering and means the process of treating textiles with a liquid composition, e.g. a wash liquor containing a detergent composition. The laundering process can for example be carried out using e.g. a household or an industrial washing machine or can be carried out by hand.

Odor capturing agent: The term “odor capturing agent” means a textile, paper capable of capturing or absorbing the odorous compound.

Odorous compound: The terms “odorous compound” and “odor” refer to any molecule or compound detectable to the human olfactory system. Odorous compounds are volatile compounds which may adhere to surfaces such as textiles and hard surfaces. Odorous compounds can exist as gaseous compounds and can also be present in other media such as liquid. Microbiel growth can produce odorous compounds. Other odous compounds comprise hydrogen sulfide, Methanethiol and/or dimethylsulfide present in flatulence; methanethiol, propanoic acid, isovaleric acid from foot odor, (E)-3-methyl-2-hexenoic acid, (S)-3-methyl-3-sulfanylhexan-1-ol and/or 3-hydroxy-3-methylhexanoic acid present in underarm odors.

Textile: The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, cellulose acetate fibers (tricell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used, it is intended to include the broader term textiles as well.

Visual indicating agent: The term “visual indicating agent” refers to a substance, a composition or a material that gives a visual indication when an odorous compound is present in a sufficient concentration. The visual indicating can be a change in color of the visual indication agent, which color change is visible to human eye. Examples of visual indicating agents are Rose Bengal, Red 28, Iron chloride, phenol Red, Brillant Yellow, Bromocresol green, Chlorophenol Red, P-naphtholbenzein, Methyl Red, Shiff's test for aldehydes and Chlorophenol Red. Further examples of visual indicating agents are mentioned below. The visual indicating agent can be sensitive to odorous compounds such odorous compounds derived from microorganisms. Examples hereof are as aldehydes, E-3-methyl-2-hexenoic acid, E-2-nonenal, butyric acid hydrogen sulfide, Methanethiol and/or dimethylsulfide, methanethiol, propanoic acid, isovaleric acid, (E)-3-methyl-2-hexenoic acid, (S)-3-methyl-3-sulfanylhexan-1-ol and/or 3-hydroxy-3-methylhexanoic acid, or the visual indicating agent can be sensitive to the acidity/alkalinity of the odorous compounds.

Wash liquor: The term “wash liquor” is defined herein as liquid composition, such as a solution or mixture of at least one surfactant and water. The wash liquor can optionally comprise a builder. The wash liquor may comprise a detergent composition for laundry, a dishwashing composition or a detergent composition for cleaning hard surfaces.

### DESCRIPTION OF THE FIGURES

FIG. 1 shows a device (1) for detecting odorous compounds from a surface (2) by use of a odor capturing agent (3) comprising a visual indicating agent.

FIG. 2 shows a device (1) having several compartments (2).

### DETAILED DESCRIPTION OF THE INVENTION

Odorous compound can be present on surfaces such as textile on clothes or garment or hard surfaces present in kitchens, bathrooms, washing machines or dish washing machines. It is possible to detect the odorous compounds by human nose, but the inventors of the present invention have surprisingly found that it is possible to visualize the release of odorous compounds from material comprising the odorous compound. Especially for odorous compounds developed from biofilm present on a surface such as textile it is difficult to visualize the odorous compounds.

The present invention concerns an assay and a method for detecting the presence of odorous compound on a surface comprising enclosing the surface in a compartment, which compartment comprises an odor capturing agent including a visual indicating agent, wherein the surface comprises an odorous compound derived from biofilm.

The invention further concerns a method for comparing the presence of odorous compound on a first surface with the presence of an odorous compound on at least one more surface comprising:

a. Enclosing the first surface in a first compartment comprising an odor capturing agent including a visual indicating agent;

b. enclosing at least one more surface in one or more separate compartments, where each compartment comprise an odor capturing agent including a visual indicating agent;

c. comparing the color development in each compartment.

The inventor has further invented a device for detecting the presence of odorous compound on at least one surface, which device comprises at least one separate compartment, wherein each compartment is adapted to receive at least one surface and each compartment comprises an odor capturing agent including a visual indicating agent. The device can form part of a kit for detecting for odorous compounds.

Detecting for the presence of odors is often done by using expensive laboratory equipment such as gas chromatography (GC) and GC-olfractometry. Other methods for detecting for odorous compounds are using a sensorical panel with trained test persons sensing the odorous compounds. The visual indicating agent of the present invention changes color when exposed to an odorous compound. Depending on the concentration of the odorous compound, the color change will be observed when the odor is present. The present invention is cheaper and easier to conduct than the know methods for detecting of odorous compounds.

The visual indicating agent is typically color-sensitive to at least one odors selected from the group comprising body odor, foot odor, garbage odor, urinary odor, feces odor, tobacco odor, raw meat odor, other common household odors such as bathroom, pet and cooking odors, mercaptans (or thiols), amines, ammonia, sulfur, sulfides, hydrogen sulphide, sulfur degradation products, aliphatic acids, isovaleric acid, butyric acid and acetic acid.

The visual indicating agent can be sensitive to odorous compounds such odorous compounds derived from microorganisms. Other odous compounds comprise hydrogen sulfide, Methanethiol and/or dimethylsulfide present in flatulence; methanethiol, propanoic acid, isovaleric acid from foot odor, (E)-3-methyl-2-hexenoic acid, (S)-3-methyl-3-sulfanylhexan-1-ol and/or 3-hydroxy-3-methylhexanoic acid present in underarm odors.

Odorous compounds can also be derived from protein, lipid, carbohydrate, microorganisms, biofilm and/or mixtures thereof.

The present invention is especially good at detecting odorous compounds released from surfaces present in laundry or dishwashing environment. In one embodiment of the invention, the odorous compound is developed from microorganisms present in dishwashing machines, washing machines, dishware and/or on laundry items. Example of such microorganisms are Brevundimonas sp. Acinetobacter sp., Aeromicrobium sp., Microbacterium sp., Micrococcus luteus, Pseudomonas sp., Staphylococcus epidermidis, and Stenotrophomonas sp. The present invention is very good at detecting odorous compounds released from biofilm. The biofilm may comprise organic and/or inorganic material, or the biofilm may comprise material secreted from human or animal body such as blood, sweat, lipid, grease, sebum, drool, vomit, cells, microorganisms, odor, DNA and/or mixtures hereof. In one embodiment of the invention the surface comprises soil or a stain, e.g. in addition to the surface comprising a biofilm.

Suitable visual indicating agents are selected from Rose Bengal, Red 28, Iron chloride, phenol Red, Brillant Yellow, Bromocresol green, Chlorophenol Red, P-naphtholbenzein, Methyl Red, Shill's test for aldehydes, Chlorophenol Red, neutral red, 3-nitrophenol, brilliant yellow, chlorophenol red, Rose Bengal dye, D & C red 28 dye, 4, 4′-bis (dimethylamino)-benzhydrol (BDMB or Michler's hydro) (MH)), methyl red, methyl violet, methyl orange, bromocresol mauve, Acid Blue 80, blue dye Calcocid Blue 2G, ethyl red, bromophenol blue, bromocresol green, crystal violet, cresol red, thymol blue, erythrosine B, 2, 4-dinitrophenol, Eriochrome Tm Black T, alizarin, bromothymol blue, phenol red, m-nitrophenol, o-cresolphthalein, thymolphthalein, alizarin Yellow Reller, cobalt salts and complexes, copper salts and complexes, copper phenanthroline complexes and iron salts and complexes.

Additional indicating agents are those represented by the following general formula (I) or (II):

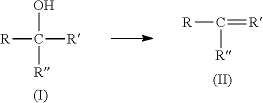

where R, R′ and R″ are as shown in Table 1:

TABLE 1
Indicating agents having the general formula (I) or (II)

| Indicating | | | | Indicating Agent |
|---|---|---|---|---|
| Agent | R | R″ | R′ | for |
| Michler's Hydrol | H | (CH₃)₂NC₆H₄ | (CH₃)₂NC₆H₄ | Thiols, Mercaptans, |
| (MH) | | | | Ammonia, Amines, |
| | | | | Diamines and |
| | | | | Polyamines |
| Pararosaniline | (NH₂)C₆H₄ | (NH₂)C₆H₄ | (NH₂)C₆H₄ | Ammonia, Amines, |
| (PAB) | | | | Diamines and |
| | | | | Polyamines |
| Alpha- naphtholbenzein (ANB) | C₆H₅ | 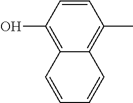 | 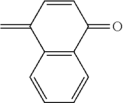 | Ammonia, Amines, Diamines and Polyamines |
| Naphthochrome Green (NCG) | C₆H₅ | 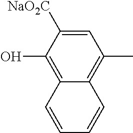 | 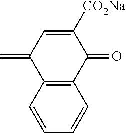 | Ammonia, Amines, Diamines and Polyamines |

The odor capturering agent is typically a filtering paper, a textile, a liquid, a gel or a paste. The visual indicating agent are applied to or integrated in the odor capturing agent. Other examples of odor capturering agents are activated charcoal, sodium bicarbonate, clay, zeolites, silicates, starches, ion exchange resins, cyclodextrins, molecular sieves or high surface area materials such as nanoparticles.

In some instances, the visual indicating agent and odor capturing agent may be the same agent. For example, BDMB may be used as both the odor capturing agent and the visual indicating agent for sulfur, amine and ammonia odors.

The odor capturing agent includes a visual indicating agent so that the visual indicating agent may be applied to the odor capturing agent in solution and allowed to dry so that a dried residue remains on the odor capturing agent. As used herein, the term solution refers to the indicating agent in a liquid such as water, an aqueous solution, alcohol, toluene and the like.

The odor capturing agent may include a single zone with a concentration of the visual indicating agent such that the visual indicating agent and hence the zone will change color to indicate that odorous compounds are detected.

However, as the concentration of the visual indicating agent and the amount of odorous compounds are the major factors determining the time in which the visual indicating agent takes to change color, the use of zones having different concentrations of the indicating agent allows a graduated scale to be produced that would indicate how much odor the odor capture agent captures. The scale may be in the form of a linear scale, a circular scale, a collection of dots, text and so forth.

In the examples which follow, a color-changing visual indicating agent was dissolved in a solvent (e. g. water, alcohol or acetonitrile) to give a concentration in the range of from 1 mg/ml to 100 mg/ml. The solution was then applied to a odor capturing agent (e. g. cellulose, cotton, nonwoven, glass fiber) by one of the following methods: (a) dropping known amounts of the solution onto the surface of the odor capturing agent, allowing the liquid to wet the odor capturing agent, and then allowing the odor capturing agent to air-dry; (b) dipping the odor capturing agent into the solution, nipping it to remove the excess solution and then air-drying or oven-drying the substrate in a low temperature oven; (c) spraying the solution onto the odor capturing agent and allowing it to dry; or (d) printing the solution onto the odor capturing agent by flexographic, off-set or inkjet printing.

In one embodiment of the invention, the compartment comprising the odor capturing agent including the visual indicating agent is hermetically sealed. The compartment is hermetically sealed for at least 8 hours, 9 hours, 10 hours, 11 hours, 12 hours, 13 hours, 14 hours, 15 hours, 16 hours, 17 hours, 18 hours, 19 hours or 20 hours.

The assay can be used for detecting the presence of odorous compounds present on laundry items, dishware or hard surfaces. The hard surface can be the interior of a washing machine or an automatic dishwashing machine. The interior of dishwashing machines or laundry washing machines may be subject to growth of biofilm. The growth and proliferation of microbes in a these machines generally occurs from exposure to prolonged warm, moist environments which may contain soap residue and clothing or food residues. This environment leads to the development of undesirable odors and biofilm. The present invention can be used to detect odorous compounds present in such environments. In one embodiment the compartment in which the surface is tested is the interior of a dishwashing machines or laundry washing machines. The test can be performed by placing the odor capturing agent including the visual indicating agent in the interior of the dishwashing machines or laundry washing machines and optionally close the door/lid.

In one embodiment, the surface to be tested have been washed in a washing machine or a dishwashing machine. The surface may be a textile or a dishware. Detection of odorous compounds on the surface can be tested before the surface is washed and/or after the surface has been washed. In one embodiment of the invention, a surface comprising a biofilm is washed together with one or more other clean surfaces (which do not comprise a biofilm). Detection of odorous compounds are then detected after wash of the first and/or the clean surfaces in order to detect the redeposition of biofilm material on the clean surfaces.

In another embodiment, clean surfaces are washed in dishwashing machines or laundry washing machines in order to detect if odorous compounds from the dishwashing machines or laundry washing machines are deposited on the clean surface.

In one embodiment, the invention further concerns a device for detecting the presence of odorous compound on at least one surface, which device comprises at least one separate compartment, wherein the at least one separate compartment is adapted to receive at least one surface and each separate compartment comprises an odor capturing agent including a visual indicating agent.

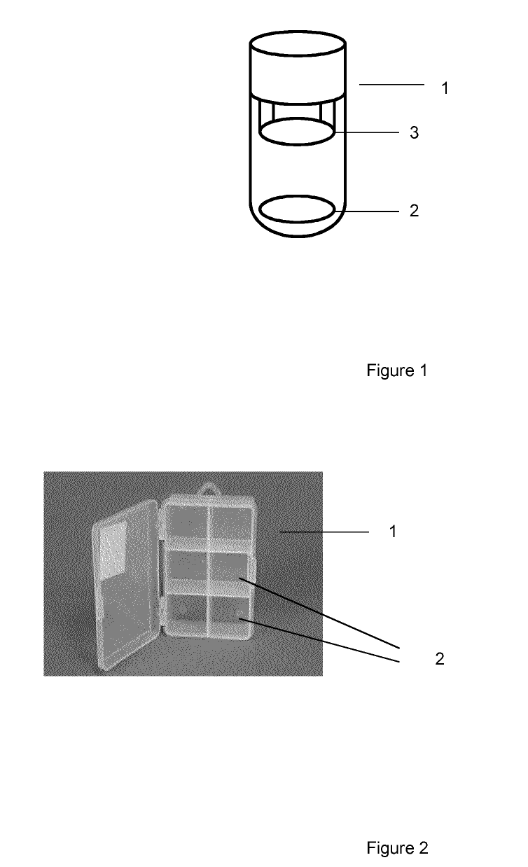

One embodiment of the device is illustrated in FIG. 1 showing a device (1) for detecting odorous compounds from a surface (2) by use of a odor capturing agent (3) comprising a visual indicating agent.

The device can comprise only one compartment adapted to receive at least one surface, which compartment comprises an odor capturing agent including a visual indicating agent. In one embodiment of the invention two compartments are used for detecting the presence of odorous compound on two different surfaces. Each compartment comprise an odor capturing agent including a visual indicating agent and the time to color change of the visual indicating agent can be determined for each of the surfaces. In one embodiment more than two compartments are used to test more than two surface for odorous compounds. In one embodiment three, four, five, six, seven, eight, nine, ten, eleven, twelve or even more compartments are used to test for odorous compounds This enables the person skilled in the art to compare two or more surface for odorous compounds. The skilled person can either start the test for all the surfaces simultaneously or use a timer to measure the number of minutes to a specific degree of color change.

In one embodiment, the device has at least two separate compartments, where each compartment comprise an odor capturing agent including a visual indicating agent. In one embodiment the device has at least three, four, five, six, seven, eight, nine, ten, eleven, twelve or even more separate compartments. In one embodiment the device is a microtiterplate.

One embodiment of the device is illustrated in FIG. 2, which shows a device (1) having several compartments (2).

In one embodiment, the intensity of the color of the visual indicating agent is measured and quantified. Light reflectance evaluations of the surfaces can done using a Macbeth Color Eye 7000 reflectance spectrophotometer with very small aperture. The measurements can be made without UV in the incident light and remission at 460 nm can be extracted.

In one embodiment of the invention, the compartment is formed as a dome or a bell that covers or enclose the surface to be tested and the odor capturing agent including the visual indicating agent.

In one embodiment of the invention, at least a part of the compartment is transparent. This enables the person using the assay to see the color change of the visual indicator agent. In one embodiment the full surface of each of the compartments are transparent.

In one embodiment, the invention concerns a kit for detecting for odorous compounds, where the kit comprises at least one device. The kit may further comprise a timer, a stop watch and/or a recording device for taking pictures/films or measuring the color intensity of the visual indicator agent.

The invention is further summarized in the following paragraphs:
1. Assay for detecting the presence of odorous compound on a surface comprising enclosing the surface in a compartment, which compartment comprises an odor capturing agent including a visual indicating agent, wherein the surface comprises an odorous compound derived from biofilm.
2. Assay according to paragraph 1, wherein the visual indicating agent is selected from the group consisting of Rose Bengal, Red 28, Iron chloride, phenol Red, Brillant Yellow, Bromocresol green, Chlorophenol Red, P-naphtholbenzein, Methyl Red, Shill's test for aldehydes, Chlorophenol Red, neutral red, 3-nitrophenol, brilliant yellow, chlorophenol red, Rose Bengal dye, D & C red 28 dye, 4, 4′-bis (dimethylamino)-benzhydrol, methyl red, methyl violet, methyl orange, bromocresol mauve, Acid Blue 80, blue dye Calcocid Blue 2G, ethyl red, bromophenol blue, bromocresol green, crystal violet, cresol red, thymol blue, erythrosine B, 2, 4-dinitrophenol, Eriochrome Black T, alizarin, bromothymol blue, phenol red, m-nitrophenol, o-cresolphthalein, thymolphthalein, alizarin Yellow Reller, cobalt salts and complexes, copper salts and complexes, copper phenanthroline complexes and iron salts and complexes thereof.
3. Assay according to paragraph 1, wherein the visual indicating agent is selected from formula (I) or (II):

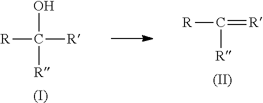

where R, R′ and R″ are shown in table 1.
4. Assay according to any of the preceding paragraph, wherein the surface in addition to the odorous compounds from biofilm comprises an odorous compound derived from protein, lipid, carbohydrate, microorganisms and/or mixtures thereof.
5. Assay according to paragraph 4, wherein the odorous compound comprises aldehydes, E-2-nonenal, hydrogen sulfide, methanethiol, dimethylsulfide, methanethiol, propanoic acid, isovaleric acid, (E)-3-methyl-2-hexenoic acid, (S)-3-methyl-3-sulfanylhexan-1-ol and/or 3-hydroxy-3-methylhexanoic acid.
6. Assay according to any of paragraphs 4-5, wherein the biofilm comprises microorganisms present in dishwashing machines, washing machines, dishware and/or on laundry items.
7. Assay according to any of paragraphs 4-6, wherein the microorganism is selected from the group consisting of Brevundimonasl sp. Acinetobacter sp., Aeromicrobium sp., Microbacterium sp., Micrococcus luteus, Pseudomonas sp., Staphylococcus epidermidis, and Stenotrophomonas sp.
8. Assay according to any of paragraphs 4-7, wherein the microorganism is Brevundimonas sp.
9. Assay according to paragraphs 4-8, wherein the biofilm comprises organic and/or inorganic material.
10. Assay according to paragraph 9, wherein the biofilm comprises material secreted from human or animal body such as blood, sweat, lipid, grease, sebum, drool, vomit, cells, microorganisms, odor, DNA and/or mixtures hereof.
11. Assay according to any of the preceding paragraphs, wherein the surface comprises soil or a stain.
12. Assay according to any of the preceding paragraphs, wherein the odor capturing agent is a filtering paper, a textile, a liquid, a gel, a paste, charcoal, sodium bicarbonate, clay, zeolites, silicates, starches, ion exchange resins, cyclodextrins, molecular sieves or high surface area materials such as nanoparticles.
13. Assay according to any of the preceding paragraphs, wherein the surface is present on a laundry item, a hard surface or a dishware.
14. Assay according to any of the preceding paragraphs, wherein at least a part of the compartment is transparent.
15. Assay according to paragraphs 14, wherein the full compartment is transparent.
16. Assay according to any of paragraphs 14-15, wherein the odor capturing agent is present in the headspace of the surface to be tested.
17. Assay according to any of the preceding paragraphs, wherein the visual indicating agent changes color.
18. Assay according to paragraph 17, wherein the color change is visible by human eye.
19. Method for detecting the presence of odorous compound on a surface comprising enclosing the surface in a compartment, which compartment comprises an odor capturing agent including a visual indicating agent, wherein the surface comprises an odorous compound derived from biofilm.
20. Method according to paragraph 19, wherein the visual indicating agent is selected from the group consisting of Rose Bengal, Red 28, Iron chloride, phenol Red, Brillant Yellow, Bromocresol green, Chlorophenol Red, P-naphtholbenzein, Methyl Red, Shill's test for aldehydes, Chlorophenol Red, neutral red, 3-nitrophenol, brilliant yellow, chlorophenol red, Rose Bengal dye, D & C red 28 dye, 4, 4′-bis (dimethylamino)-benzhydrol, methyl red, methyl violet, methyl orange, bromocresol mauve, Acid Blue 80, blue dye Calcocid Blue 2G, ethyl red, bromophenol blue, bromocresol green, crystal violet, cresol red, thymol blue, erythrosine B, 2, 4-dinitrophenol, Eriochrome Black T, alizarin, bromothymol blue, phenol red, m-nitrophenol, o-cresolphthalein, thymolphthalein, alizarin Yellow Reller, cobalt salts and complexes, copper salts and complexes, copper phenanthroline complexes and iron salts and complexes thereof.
21. Method according to paragraph 19, wherein the visual indicating agent is selected from formula (I) or (II):

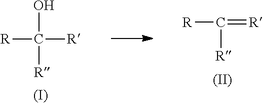

where R, R′ and R″ are shown in table 1.
22. Method according to any of the preceding method paragraph, wherein the surface in addition to the odorous compound derived from biofilm comprises an odorous compound derived from protein, lipid, carbohydrate, microorganisms and/or mixtures thereof.
23. Method according to any of the preceding method paragraphs, wherein the odorous compound comprises aldehydes, E-2-nonenal, hydrogen sulfide, methanethiol, dimethylsulfide, methanethiol, propanoic acid, isovaleric acid, (E)-3-methyl-2-hexenoic acid, (S)-3-methyl-3-sulfanylhexan-1-ol and/or 3-hydroxy-3-methylhexanoic acid.
24. Method according to any of paragraphs 22-23, wherein the biofilm comprises microorganisms present in dishwashing machines, washing machines, dishware and/or on laundry items.
25. Method according to any of paragraphs 22-24, wherein the microorganism is selected from the group consisting of Brevundimonasl sp. Acinetobacter sp., Aeromicrobium sp., Microbacterium sp., Micrococcus luteus, Pseudomonas sp., Staphylococcus epidermidis, and Stenotrophomonas sp.
26. Method according to any of paragraphs 22-25, wherein the microorganism is Brevundimonas sp.
27. Method according to paragraphs 22-26, wherein the biofilm comprises organic and/or inorganic material.
28. Method according to paragraphs 22-27, wherein the biofilm comprises material secreted from human or animal body such as blood, sweat, lipid, grease, sebum, drool, vomit, cells, microorganisms, odor, DNA and/or mixtures hereof.
29. Method according to any of paragraphs 22-28, wherein the surface comprises soil or a stain.
30. Method according to any of the preceding method paragraphs, wherein the odor capturing agent is a filtering paper, a textile, a liquid, a gel, a paste, charcoal, sodium bicarbonate, clay, zeolites, silicates, starches, ion exchange resins, cyclodextrins, molecular sieves or high surface area materials such as nanoparticles.
31. Method according to any of the preceding method paragraphs, wherein the surface is present on a laundry item, a hard surface or a dishware.
32. Method according to any of the preceding method paragraphs, wherein at least a part of the compartment is transparent.
33. Method according to paragraph 32, wherein the full compartment is transparent.
34. Method according to any of paragraphs 32-33, wherein the odor capturing agent is present in the headspace of the surface to be tested.
35. Method according to any of the preceding method paragraphs, wherein the visual indicating agent changes color.
36. Method according to paragraph 35, wherein the color change is visible by human eye.
37. A method for comparing the presence of odorous compound on a first surface with the presence of an odorous compound on at least one more surface comprising:
   a. Enclosing the first surface in a first compartment comprising an odor capturing agent including a visual indicating agent;
   b. enclosing at least one more surface in one or more separate compartments, where each compartment comprise an odor capturing agent including a visual indicating agent;
   c. comparing the color development in each compartment,
      wherein the first surface and/or the at least one more surface comprises an odorous compound derived from biofilm.
38. Method according to paragraph 37, wherein the visual indicating agent is selected from the group consisting of Rose Bengal, Red 28, Iron chloride, phenol Red, Brillant Yellow, Bromocresol green, Chlorophenol Red, P-naphtholbenzein, Methyl Red, Shill's test for aldehydes, Chlorophenol Red, neutral red, 3-nitrophenol, brilliant yellow, chlorophenol red, Rose Bengal dye, D & C red 28 dye, 4, 4′-bis (dimethylamino)-benzhydrol, methyl red, methyl violet, methyl orange, bromocresol mauve, Acid Blue 80, blue dye Calcocid Blue 2G, ethyl red, bromophenol blue, bromocresol green, crystal violet, cresol red, thymol blue, erythrosine B, 2, 4-dinitrophenol, Eriochrome Black T, alizarin, bromothymol blue, phenol red, m-nitrophenol, o-cresolphthalein, thymolphthalein, alizarin Yellow Reller, cobalt salts and complexes, copper salts and complexes, copper phenanthroline complexes and iron salts and complexes thereof.
39. Method according to paragraph 37, wherein the visual indicating agent is selected from formula (I) or (II):

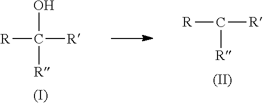

where R, R′ and R″ are shown in table 1.
40. Method according to any of paragraphs 37-39, wherein the first surface and/or the at least one more surface in addition to the odorous compound derived from biofilm comprises an odorous compound derived from protein, lipid, carbohydrate, microorganisms and/or mixtures thereof.
41. Method according to paragraph 40, wherein the odorous compound comprises aldehydes, E-2-nonenal, hydrogen sulfide, methanethiol, dimethylsulfide, methanethiol, propanoic acid, isovaleric acid, (E)-3-methyl-2-hexenoic acid, (S)-3-methyl-3-sulfanylhexan-1-ol and/or 3-hydroxy-3-methylhexanoic acid.
42. Method according to any of paragraphs 40-41, wherein the biofilm comprises microorganisms present in dishwashing machines, washing machines, dishware and/or on laundry items.
43. Method according to any of paragraphs 40-42, wherein the microorganism is selected from the group consisting of Brevundimonasl sp. Acinetobacter sp., Aeromicrobium sp., Microbacterium sp., Micrococcus luteus, Pseudomonas sp., Staphylococcus epidermidis, and Stenotrophomonas sp.
44. Method according to any of paragraphs 40-43, wherein the microorganism is Brevundimonas sp.
45. Method according to paragraphs 40-44, wherein the biofilm comprises organic and/or inorganic material.
46. Method according to paragraphs 40-45, wherein the biofilm comprises material secreted from human or animal body such as blood, sweat, lipid, grease, sebum, drool, vomit, cells, microorganisms, odor, DNA and/or mixtures hereof.
47. Method according to any of paragraphs 40-46, wherein the first and the at least one more surface are similar.
48. Method according to any of paragraphs 40-47, wherein the first surface comprises a biofilm and have been washed in a wash liquor and the at least one more surface have been washed in the same wash liquor, wherein the wash liquor optionally comprises an enzyme.
49. Method according to any of paragraphs 40-48, wherein the surface comprises soil or a stain.
50. Method according to any of paragraphs 37-49, wherein the odor capturing agent is a filtering paper, a textile, a liquid, a gel, a paste, charcoal, sodium bicarbonate, clay, zeolites, silicates, starches, ion exchange resins, cyclodextrins, molecular sieves or high surface area materials such as nanoparticles.
51. Method according to any of paragraphs 37-50, wherein the first surface is present on a laundry item, a hard surface or a dishware.
52. Method according to any of paragraphs 37-51, wherein at least a part of the compartment is transparent.
53. Method according to paragraphs 52, wherein the full compartment is transparent.
54. Method according to any of paragraphs 52-53, wherein the odor capturing agent is present in the headspace of the surface to be tested.
55. Method according to any of paragraphs 37-54, wherein the visual indicating agent changes color.
56. Method according to paragraph 55, wherein the color change is visible by human eye.
57. Method according to any of paragraphs 37-56, wherein the first surface is washed with a first wash liquor and the at least one more surface is washed with a wash liquor different from the first wash liquor.
58. Method according to any of paragraphs 37-56, wherein the first surface is not washed and the at least one more surface is washed with same or different wash liquors.
59. Method according to any of paragraphs 57-58, wherein the first wash liquor comprises at least one enzyme.
60. Method according to any of paragraphs 37-59, wherein the color development of the visual indicating agent is recorded.
61. Device for detecting the presence of odorous compound on at least one surface, which device comprises at least one separate compartment, wherein the at least one separate compartment is adapted to receive at least one surface and each separate compartment comprises an odor capturing agent including a visual indicating agent.
62. Device according to paragraph 61, wherein at least a part of the compartment is transparent.
63. Device according to any of paragraphs 61-62, wherein at least part of each compartment is transparent.
64. Device according to any of paragraphs 61-63, wherein the full compartment is transparent.
65. Device according to any of paragraphs 61-64, wherein the visual indicating agent changes color.
66. Device according to any of paragraphs 61-65, wherein the visual indicating agent is selected from the group consisting of Rose Bengal, Red 28, Iron chloride, phenol Red, Brillant Yellow, Bromocresol green, Chlorophenol Red, P-naphtholbenzein, Methyl Red, Shill's test for aldehydes, Chlorophenol Red, neutral red, 3-nitrophenol, brilliant yellow, chlorophenol red, Rose Bengal dye, D & C red 28 dye, 4, 4′-bis (dimethylamino)-benzhydrol, methyl red, methyl violet, methyl orange, bromocresol mauve, Acid Blue 80, blue dye Calcocid Blue 2G, ethyl red, bromophenol blue, bromocresol green, crystal violet, cresol red, thymol blue, erythrosine B, 2, 4-dinitrophenol, Eriochrome Black T, alizarin, bromothymol blue, phenol red, m-nitrophenol, o-cresolphthalein, thymolphthalein, alizarin Yellow Reller, cobalt salts and complexes, copper salts and complexes, copper phenanthroline complexes and iron salts and complexes thereof.
67. Device according to any of paragraphs 61-65, wherein the visual indicating agent is selected from formula (I) or (II):

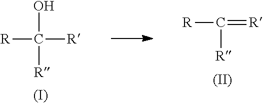

where R, R′ and R″ are shown in table 1.
68. Device according to any of paragraphs 61-67, wherein the odor capturing agent is a filtering paper, a textile, a liquid, a gel, a paste, charcoal, sodium bicarbonate, clay, zeolites, silicates, starches, ion exchange resins, cyclodextrins, molecular sieves or high surface area materials such as nanoparticles.
69. Kit for detecting for an odorous compound comprising at least one device.
70. Kit according to paragraph 69, wherein the kit further comprise a stop watch, a timer and/or a recording device.

### Assays and Compositions

### Composition of Model Detergent A (Liquid)

Ingredients: 12% LAS, 11% AEO Biosoft N25-7 (NI), 7% AEOS (SLES), 6% MPG (monopropylene glycol), 3% ethanol, 3% TEA, 2.75% cocoa soap, 2.75% soya soap, 2% glycerol, 2% sodium hydroxide, 2% sodium citrate, 1% sodium formiate, 0.2% DTMPA and 0.2% PCA (all percentages are w/w).

### Composition of Tide Liquid Original

Ingredients: Linear alkylbenzene sulfonate, propylene glycol, citric acid, sodium hydroxide, borax, ethanolamine, ethanol, alcohol sulfate, polyethyleneimine ethoxylate, sodium fatty acids, diquaternium ethoxysulfate, protease, diethylene glycol, laureth-9, alkyldimethylamine oxide, fragrance, amylase, disodium diaminostilbene disulfonate, DTPA, sodium formate, calcium formate, polyethylene glycol 4000, mannanase, Liquitint™ Blue, dimethicone.

### Assay I

### Washing Test in Beaker

500 ml deionized water was added in each beaker. The water was heated to 40° C. and a liquid composition (e.g. wash liquor) was added and stirred for 5 minutes. If an enzyme should be included in the testing, the enzyme was added into one beaker together with the liquid composition. 6 swatches were added into each beaker, and stirred for 15 minutes. The swatches were rinsed in tap water, and results evaluated.

### Assay II

### Terg-O-Tometer (TOM) Wash Assay

The Tergo-To-Meter (TOM) is a medium scale model wash system that can be applied to test 12 different wash conditions simultaneously. A TOM is basically a large temperature controlled water bath with up to 12 open metal beakers submerged into it. Each beaker constitutes one small top loader style washing machine and during an experiment, each of them will contain a solution of a specific detergent/enzyme system and the soiled and unsoiled fabrics its performance is tested on. Mechanical stress is achieved by a rotating stirring arm, which stirs the liquid within each beaker. Because the TOM beakers have no lid, it is possible to withdraw samples during a TOM experiment and assay for information on-line during wash.

The TOM model wash system is mainly used in medium scale testing of detergents and enzymes at US or LA/AP wash conditions. In a TOM experiment, factors such as the ballast to soil ratio and the fabric to wash liquor ratio can be varied. Therefore, the TOM provides the link between small scale experiments, such as AMSA and mini-wash, and the more time consuming full scale experiments in top loader washing machines.

Equipment: The water bath with 12 steel beakers and 1 rotating arm per beaker with capacity of 500 or 1200 mL of detergent solution. Temperature ranges from 5 to 80° C. The water bath has to be filled up with deionised water. Rotational speed can be set up to 70 to 120 rpm/min.

Set temperature in the Terg-O-Tometer and start the rotation in the water bath. Wait for the temperature to adjust (tolerance is +/−0.5° C.)

All beakers shall be clean and without traces of prior test material.

Prepare wash solution with desired amount of detergent, temperature and water hardness in a bucket. Let detergent dissolve during magnet stirring for 10 min. Wash solution shall be used within 30 to 60 min after preparation.

Add 1000 ml wash solution into a TOM beaker

Start agitation at 60 rpm and optionally add enzymes to the beaker.

Sprinkle the swatches into the beaker and then the ballast load.

Time measurement start when the swatches and ballast are added to the beaker.

Wash for 15 minutes

Stop agitation

Transfer the wash load from TOM beaker to a sieve and rinse with cold tap water for 5 minutes, rinse swatches washed with or without enzyme separately. Press gently the water out by hand and place the test swatches on a tray covered with a paper. Add another paper on top of the swatches. Let the swatches dry overnight.

### Assay III

The odor capturing agent including the visual indicating agent is spread out flat and allowed to air dry at room temperature overnight. Light reflectance evaluations of the odor capturing agent including the visual indicating agent were done using a Macbeth Color Eye 7000 reflectance spectrophotometer with very small aperture. The measurements were made without UV in the incident light and remission at 460 nm was extracted.

### Assay IV

### Full Scale Wash:

This is the test method used to test under full scale wash under EU conditions, where real laundry items can be washed. The real items (e.g. Shirts) are added to each wash together with a liquid composition e.g. a detergent and optionally comprising an enzyme. The enzymes are added on basis of weight percent of the detergent dosage in each wash. After wash, the real items are dried overnight.

### Equipment Used:

Washing machine: Miele Softtronic W2445
Water meters and automatically data collection system

### For the Preparation and Adjustment of Water Hardness the Following Ingredients are Needed:

Calcium chloride (CaCl₂.2H₂O)
Magnesium chloride (MgCL₂.6H₂O)
Sodium Hydrogen Carbonate (NaHCO₃)

### Wash Conditions:

Temperature: 40° C.
Washing programme: Normal cotton wash without pre-wash: “Cottons”.
Water level 12-13 L with “water plus”
Water hardness: Standard EU conditions: 15° dH, Ca2+:Mg2+:HCO3=4:1:7.5

### Detailed Steps to Carry Out Full Scale Wash Trial

1. Select wash program as in study plan.
2. The detergent and Enzyme are placed in the wash drum in a “washing ball” (both liquid and powder detergents). Place it at the bottom.
3. Place the real items in the wash drum.
4. Start digital water meter
5. Start the washer by pressing the knob START
6. After wash, take out real items and dry them at the room temperature overnight.

### Drying Procedure

Hang the items in line and dry at room temperature overnight.

### EXAMPLES

### Example 1

### Preparing a Biofilm Comprising Brevundimonas sp.

### Isolating Laundry Specific Bacterial Strains

One strain of Brevundimonas sp. isolated from laundry was used in the present example.

The Brevundimonas sp. was isolated during a study, where the bacterial diversity in laundry after washing at 15, 40 and 60° C., respectively, was investigated. The study was conducted on laundry collected from Danish households. For each wash, 20 g of laundry items (tea towel, towel, dish cloth, bib, T-shirt armpit, T-shirt collar, socks) in the range 4:3:2:2:1:1:1 was used. Washing was performed in a Laundr-O-Meter (LOM) at 15, 40 or 60° C. For washing at 15 and 40° C., Ariel Sensitive White & Color was used, whereas WFK IEC-A* model detergent was used for washing at 60° C. Ariel Sensitive White & Color was prepared by weighing out 5.1 g and adding tap water up to 1000 ml followed by stirring for 5 minutes. WFK IEC-A* model detergent (which is available from WFK Testgewebe GmbH) was prepared by weighing out 5 g and adding tap water up to 1300 ml followed by stirring for 15 min. Washing was performed for 1 hour at 15, 40 and 60° C., respectively, followed by 2 times rinsing with tap water for 20 min at 15° C.

Laundry was sampled immediately after washing at 15, 40 and 60° C., respectively. Twenty grams of laundry was added 0.9% (w/v) NaCl (1.06404; Merck, Damstadt, Germany) with 0.5% (w/w) tween 80 to yield a 1:10 dilution in stomacher bag. The mixture was homogenized using a Stomacher for 2 minutes at medium speed. After homogenization, ten-fold dilutions were prepared in 0.9% (w/v) NaCl. Bacteria were enumerated on Tryptone Soya Agar (TSA) (CM0129, Oxoid, Basingstoke, Hampshire, UK) incubated aerobically at 30° C. for 5-7 days. To suppress growth of yeast and moulds, 0.2% sorbic acid (359769, Sigma) and 0.1% cycloheximide (18079; Sigma) were added. Bacterial colonies were selected from countable plates and purified by restreaking twice on TSA. For long time storage, purified isolates were stored at −80° C. in TSB containing 20% (w/v) glycerol (49779; Sigma).

### Preparation of Brevundimonas Biofilm Swatch

Brevundimonas sp. was pre-grown on Tryptone Soya Agar (TSA) (pH 7.3) (CM0131; Oxoid Ltd, Basingstoke, UK) for 2-5 days at 30° C. From a single colony, a loop-full was transferred to 10 mL of TSB and incubated for 20 hours at 30° C. with shaking (240 rpm). After propagation, Brevundimonas sp. was pelleted by centrifugation (Sigma Laboratory Centrifuge 6K15) (3000 g at 21° C. in 7 min) and resuspended in 10 mL of TSB diluted twice with water. Optical density (OD) at 600 nm was measured using a spectophometer (POLARstar Omega (BMG Labtech, Ortenberg, Germany). Fresh TSB diluted twice with water was inoculated to an OD600 nm of 0.03, and 20 mL was added to a petridish, in which 5 cm×5 cm swatch of sterile Polyester WFK30A was placed. After incubation (24 h at 15° C. with shaking (75 rpm), swatches were rinsed twice with 0.9% (w/v) NaCl. Swatches were washed in TOM either directly after rinsing or after drying for 24 hours in a LAF bench.

### Example 2

Example 2 demonstrated the detection of odorous compounds in assays with different visual indicating agents used in the assay.

TABLE 2

| | Reacting | | | |
|---|---|---|---|---|
| | with & | | | |
| Visual | visual | | | |
| indicating | transition | Color | | |
| agent | interval | change | Solvent | Conc. |
| Rose Bengal | Sulfur | red to white | water | 0.1% |
| | compounds | | | |
| Red 28 | Sulfur | red to white | water | 0.1% |
| | compounds | | | |
| Iron chloride | Sulfur | Yellow/brown | water | 1% |
| | compounds | to coloress | | |
| | and amines | or | | |
| | | brown/black | | |
| Phenol red | pH (6.8-8.2) | Yellow to red | ethanol | 0.1% |
| Brillant Yellow | pH (6.8-8.2) | Yellow to | water | 0.2% |
| | | reddish- | | |
| | | orange | | |
| Bromocresol | pH (3.8-5.4) | Yellow to | ethanol | 0.05% |
| green | | blue | | |
| Chlorophenol | pH (4.8-6.7) | Yellow to | water | 1% |
| Red | | violet | | |
| P- | pH (8.2-10.0) | Orange to | ethanol | 0.05% |
| naphtholbenzein | | green | | |
| Methyl Red | pH (4.4-6.2) | Red to | ethanol | 0.1% |
| | | yellow | | |
| Shiff's test for | Aldehydes | Colorless to | As is | 100% |
| aldehydes | | magenta | | |
| Chlorophenol | pH (4.8 to 6.7) | Yellow to | water | 0.1% |
| Red | | violet | | |

### Iron Chloride Used a Visual Indicating Agent

The visual indicating agent iron chloride solution was made according to above table 2. 50 μL of the visual indicating agent in solution was added to the filter paper (Ø 2 cm) and dried overnight in fume hood to form a odor capturing agent including a visual indicating agent. 3 cotton biofilm swatches (preparation described in example 1) were placed in the bottom of the compartments in the test glass as illustrated in FIG. 1. 200 μL of sterile deionized water was mixed with 100 μl of 50% TSB containing Brevundimonas (OD₆₀₀=0.03 in 50% TSB). As a control 3 clean cotton swatches were placed in the bottom of another test glass. 200 μL of sterile deionized water was mixed with 100 μl of 50% TSB and added to the test glass. The setup as described in FIG. 1 was prepared and the test glasses incubated at 37° C.

### Phenol Red Used a Visual Indicating Agent

The visual indicating agent Phenol red solution was prepared according to above table 2. 50 μL of the visual indicating agent in solution was added to the filter paper (Ø 2 cm) and dried overnight in fume hood to form a odor capturing agent including a visual indicating agent. 5 biofilm cotton or polyester swatches (Ø2 cm, preparation described in 1) were placed in the bottom of the test glass and 500 μL of 17% TSB. As a control 5 clean cotton or polyester swatches were placed in the bottom of a test glass and 500 μL of 17% TSB was added. The setup as described in FIG. 1 was prepared and the test glasses incubated at 37° C.

### Brilliant Yellow Used a Visual Indicating Agent

The visual indicating agent Brilliant Yellow solution was prepared according to above table 2. 50 μL of the visual indicating agent in solution was added to the filter paper (Ø 2 cm) and dried overnight in fume hood to form a odor capturing agent including a visual indicating agent. 5 biofilm cotton or polyester swatches (Ø 2 cm, preparation described in 1) were placed in the bottom of the test glass and 500 μL of 17% TSB. As a control 5 clean cotton or polyester swatches were placed in the bottom of a test glass and 500 μL of 17% TSB was added. The setup as described in FIG. 1 was prepared and the test glasses incubated at 37° C.

Bromocresol Green Reaction with 2-Methylhexanoic Acid Used a Visual Indicating Agent

The visual indicating agent bromocresol green solution was made according to above table 2. 50 μL of the indicator solution was added to the filter paper (Ø 2 cm) and dried overnight in fume hood to form a odor capturing agent including a visual indicating agent. The odor swatch was prepared with 500 μL of 10% 2-methylhexanoic acid added to 3 cotton swatches (Ø 2 cm). As a control 500 μL of deionized water was added to 3 cotton swatches (Ø 2 cm). The setup as described in FIG. 1 was prepared and the test glasses incubated at 37° C.

### Chlorophenol Red Used a Visual Indicating Agent

The visual indicating agent Chlorophenol Red solution was made according to above table 2. 50 μL of the indicator solution was added to the filter paper (Ø 2 cm) and dried overnight in fume hood to form a odor capturing agent including a visual indicating agent. The odor swatch was prepared with 500 μL of 10% 2-methylhexanoic acid added to 3 cotton swatches (Ø 2 cm). As a control 500 μL of deionized water was added to 3 cotton swatches (Ø 2 cm). The setup as described in FIG. 1 was prepared and the test glasses incubated at 37° C.

### Shiff's Test for Aldehydes Used a Visual Indicating Agent

The Shill's test solution was used as visual indication agent and was made according to above table 2. 50 μL of the indicator solution was added to the filter paper (Ø 2 cm) and dried overnight in fume hood to form a odor capturing agent including a visual indicating agent. The odor swatch was prepared with 500 μL of 0.002% nonenal added to 5 cotton swatches (Ø 2 cm). As a control 500 μL of deionized water was added to 5 cotton swatches (Ø 2 cm). The setup as described in FIG. 1 was prepared and the test glasses incubated at 37° C.

### Results:

| | Color observation end | | | |
|---|---|---|---|---|
| | of reaction/time | | | |
| | | | Odor | Odor |
| Visual | Blank | Blank | compound | compound |
| indicating | (polyester | (cotton | (polyester | (cotton |
| agent | swatch) | swatch) | swatch)/ | swatch) |
| Iron | Light Yellow/ | Light Yellow/ | Light brown | Light brown/ |
| chloride | 16 hrs | 16 hrs | 16 hrs | 16 hrs |
| Phenol Red | Yellow/ | Yellow/ | Red/ | Red/ |
| | 16 hrs | 16 hrs | 16 hrs | 16 hrs |
| Brillant | Yellow/ | Yellow/ | Orange/ | Orange/ |
| Yellow | 16 hrs | 16 hrs | 16 hrs | 16 hrs |
| Bromocresol | Light Blue/ | Light Blue/ | Green/ | Green/ |
| green | 48 hrs | 48 hrs | 48 hrs | 48 hrs |
| Chlorophenol | Pink/ | Pink/ | Yellow/ | Yellow/ |
| Red | violet/ | violet/ | brown/ | brown/ |
| | 16 hrs | 16 hrs | 16 hrs | 16 hrs |
| Shiff's test for | White/ | White/ | Light blue/ | Light blue/ |
| aldehydes | 0.5 hr | 0.5 hr | 0.5 hr | 0.5 hr |

Visual indicating agents incubated with polyester and cotton swatches with odor compounds had all changed to odour indicating color after incubation. Visual indicating agents incubated with the control clean textile did not indicate any odor development.

### Example 3

Examples of Detection of Odorous Compounds after Wash of Textile Swatches

Biofilm swatches were prepared according to 1. The swatches were washed in TOM (Assay II) using below conditions:

| Detergent (g/L) | 1.4 g/l | |
|---|---|---|
| Wash volumen | 1 | L |
| Time | 35 | min |
| Amount swatches | 3 pcs. polyester donor: Biofilm swatch | |
| per beaker 1 | 3 pcs. polyester tracer: Sterile* Wfk 30 A. | |
| | 100% white Polyester pre-washed | |
| | 3 pcs. cotton donor: Biofilm swatch | |
| | 3 pcs cotton tracer: Sterile* Wfk 10 A. | |
| | 100% white Cotton pre-washed whiteness | |
| | tracer | |
| Amount swatches | 6 pcs. Clean polyester textile: Sterile* | |
| per beaker 2 | Wfk 30 A. 100% white Polyester pre-washed | |
| | 6 pcs Clean cotton textile: Sterile* Wfk 10 A. | |
| | 100% white Cotton pre-washed whiteness tracer | |
| Water hardness | 15° | dH |
| Temperature | 30° | C. |
| RPM | 110 | rpm |
| Enzyme (ppm) | 25 | ppm |

| | |
|---|---|
| Rinse amount | 3 × 1000 ml sterile water (10 minutes rinse) |

Sterile* = All swatches has been autoclaved (121° C./20 min) before inoculation or before they are used as tracer in a wash

The detergent used is Tide Liquid Original.

### Detection of Odorous Compounds of Washed Swatches:

The visual indicating agent Phenol red in solution was prepared according to above table 2 in example 2. 50 μL of the indicator solution was added to the filter paper (Ø 2 cm) and dried overnight in fume hood to form a odor capturing agent including a visual indicating agent. 4 washed swatches (beaker 1): Polyester donor, Polyester tracer, Cotton donor and Cotton tracer were each placed in the bottom of the test glass and 500 μL of 17% TSB was added. As a control 1 washed clean textile polyester or cotton (beaker 2) were placed in the bottom of another test glass and 500 μL of 17% TSB was added. The setup as described in FIG. 1 was prepared and the test glasses (compartments) incubated at 37° C.

The following incubation results were observed:

| | Color observation end of reaction/time | | | | | |
|---|---|---|---|---|---|---|
| Visual | Pol- | Pol- | | | Clean | Clean |
| indicating | yester | yester | Cotton | Cotton | polyester | cotton |
| agent | doner | tracer | doner | tracer | textile | textile |
| Phenol | Red/ | Red/ | Red/ | Red/ | Yellow/ | Yellow/ |
| Red | 16 hrs | 16 hrs | 16 hrs | 16 hrs | 16 hrs | 16 hrs |

Visual indicating agents incubated with polyester and cotton tracers and doners were all red after incubation indicating odour development. Visual indicating agents incubated with the control clean textiles were yellow indicating that there were no odour development.

### Example 4

Examples of Detection of Odorous Compounds after Wash of Real Item T-Shirt

The real item T shirt was washed in EU full scale wash (see Assay IV) using below conditions:

| Detergent | Model detergent A (liquid) |
|---|---|
| Detergent (g/L) | 3.33 |
| Real item | Real item: T-shirt, polyester |
| | Control: Wfk 30 A. 100% white |
| | polyester pre-washed |
| Water hardness | Tap water (Denmark) |
| Temperature | 30° C. |

### Detection of Odorous Compounds of Washed Real Item T-Shirt:

The indicator Phenol red solution was prepared according to above table 2. 50 μL of the indicator solution was added to the filter paper (Ø 2 cm) and dried overnight in fume hood. The washed real item swatch (Ø 5 cm) was placed in the bottom of the test glass and 500 μL of 17% TSB was added. As a control 500 μL of 17% TSB was added to 1 polyester swatches (Ø 5 cm). The setup as described in FIG. 1 was prepared and the test glasses incubated at 37° C.

The following incubation results were observed:

| | Color observation | |
|---|---|---|
| Visual | end of reaction/time | |
| indicating | Real item | |
| agent | T shirt | Control |
| Indicator: | Red/ | Yellow/ |
| Phenol Red | 24 hrs | 24 hrs |

Visual indicating agent incubated with real item T-shirt was red after end reaction indicating odour development. Visual indicating agent incubated with control clean textile was yellow after end reaction indicating that there were no odour development.

1. Assay for detecting the presence of odorous compound on a surface comprising enclosing the surface in a compartment, which compartment comprises an odor capturing agent including a visual indicating agent, wherein the surface comprises an odorous compound derived from biofilm.

2. Assay according to claim 1, wherein the visual indicating agent is selected from the group consisting of Rose Bengal, Red 28, Iron chloride, phenol Red, Brillant Yellow, Bromocresol green, Chlorophenol Red, P-naphtholbenzein, Methyl Red, Shiff's test for aldehydes, Chlorophenol Red, neutral red, 3-nitrophenol, brilliant yellow, chlorophenol red, Rose Bengal dye, D & C red 28 dye, 4, 4′-bis (dimethylamino)-benzhydrol, methyl red, methyl violet, methyl orange, bromocresol mauve, Acid Blue 80, blue dye Calcocid Blue 2G, ethyl red, bromophenol blue, bromocresol green, crystal violet, cresol red, thymol blue, erythrosine B, 2, 4-dinitrophenol, Eriochrome Black T, alizarin, bromothymol blue, phenol red, m-nitrophenol, o-cresolphthalein, thymolphthalein, alizarin Yellow Reller, cobalt salts and complexes, copper salts and complexes, copper phenanthroline complexes and iron salts and complexes thereof.

3. Assay according to claim 1, wherein the odor capturing agent is a filtering paper, a textile, a liquid, a gel or a paste, charcoal, sodium bicarbonate, clay, zeolites, silicates, starches, ion exchange resins, cyclodextrins, molecular sieves or high surface area materials such as nanoparticles.

4. Assay according to claim 1, wherein the compartment is hermetically sealed.

5. (canceled)

6. A method for comparing the presence of odorous compound on a first surface with the presence of an odorous compound on at least one more surface comprising:
a. Enclosing the first surface in a first compartment comprising an odor capturing agent including a visual indicating agent;
b. enclosing at least one more surface in one or more separate compartments, where each compartment comprise an odor capturing agent including a visual indicating agent;
c. comparing the color development in each compartment,
wherein the first surface and/or the at least one more surface comprises an odorous compound derived from biofilm.

7. Method according to claim 6, wherein the first surface comprises a biofilm and has been washed in a first wash liquor and the at least one more surface has been washed in the same wash liquor.

8. Method according to claim 6, wherein the first surface is washed with a first wash liquor and the at least one more surface is washed with a wash liquor different from the first wash liquor.

9. Method according to claim 6, wherein the first surface is not washed and the at least one more surface is washed with same or different wash liquors.

10. Method according to claim 7, wherein the first wash liquor comprises at least one enzyme.

11. Device for detecting the presence of odorous compound on at least one surface, which device comprises at least one separate compartment, wherein the at least one separate compartment is adapted to receive at least one surface and each separate compartment comprises an odor capturing agent including a visual indicating agent.

12. Device according to claim 11, wherein at least a part of the compartment is transparent.

13. Device according to claim 11, wherein the full compartment is transparent.

14. Kit for detecting an odorous compound comprising at least one device.

15. Kit according to claim 14, wherein the kit further comprises a stop watch, a timer and/or a recording device.
